(19) United States
(12) Patent Application Publication
(10) Pub. No.: US 2007/0190001 A1
(43) Pub. Date: Aug. 16, 2007

(54) Cosmetic composition comprising an ester of polyol(s) and of fatty diacid dimer and a polyamide

(75) Inventors: Veronique Jacques, Bourg la Reine (FR); Veronique Schwartz, Paris (FR)

Correspondence Address: OLIFF & BERRIDGE, PLC, P.O. BOX 19928, ALEXANDRIA, VA, 22320, US

(73) Assignee: L'OREAL, PARIS (FR)

(21) Appl. No.: 11/705,740

(22) Filed: Feb. 14, 2007

Related U.S. Application Data
(60) Provisional application No. 60/777,578, filed on Mar. 1, 2006.

(30) Foreign Application Priority Data
Feb. 15, 2006 (FR) 06 50545

(51) Int. Cl. A61K 8/73

(52) U.S. Cl. 424/63; 424/64; 424/69; 424/70.7; 424/70.17

(57) ABSTRACT

The present invention relates to a cosmetic composition for making up and/or caring for keratin materials, combining at least one ester of polyol(s) and of fatty diacid dimer, or an ester thereof, with at least one polyamide with an average molecular mass of less than 100,000, comprising a) a polymer backbone with hydrocarbon-based repeating units containing at least one amide function, b) optionally a pendent fatty chain and/or at least one terminal fatty chain, which is optionally functionalized, containing from 6 to 120 carbon atoms, and being linked to these hydrocarbon-based units.

This non provisional application claims the benefit of French Application No. 06 50545 filed on Feb. 15, 2006 and U.S. Provisional Application No. 60/777,578 filed on Mar. 1, 2006.

The present invention relates to cosmetic compositions, intended for making up and/or caring for keratin materials, for example the skin and/or the lips, combining at least one ester of polyol(s) and of fatty diacid dimer, or an ester thereof, and at least one polyamide.

A composition according to the invention may be a makeup and/or care product intended to be applied to keratin materials such as bodily skin, for example the face and/or the lips, or the eyelashes, and may be a gloss, a lip balm, a foundation, for example cast in a dish, a skin colouring product, an eye makeup product such as a mascara, a makeup rouge, an eyeshadow, a concealer product or a lip gloss.

The present invention also relates to a process for making up the human face and/or body using a composition according to the invention.

Many cosmetic compositions exist for which gloss and/or colour effect properties of the film deposited on the skin and/or the lips are desired. These properties generally contribute towards the desired aesthetic effect.

In particular, compositions comprising a gelled or structured fatty phase may be used in lip makeup compositions of “gloss” type, which, depending on the nature of the oils of the fatty phase, may give a glossy deposit on the lips.

However, these compositions may have insufficient hardness and/or may be in a liquid or sparingly viscous form. Such a formulation requires particular attention on the part of the user as regards the storage of the container containing the composition, in order to avoid spilling it. Moreover, these compositions also require the use of special application means.

Thus, these formulations are relatively impractical to use, and the presence of additional accessories may increase their production cost.

Moreover, such compositions may also show instability over time, reflected by phase separation or syneresis.

In order to avoid these drawbacks, these compositions may be structured using agents that make them much harder, but that often result in impairment of their initial gloss.

Consequently, there is a need for cosmetic compositions that have a sufficient consistency, for example so as not to flow under their own weight, and which are, for example, compatible with application by finger, while at the same time having a fluidity that favours rapid and easy spreading on the support under consideration, for example the lips.

There is also a need for cosmetic compositions which, in addition, do not induce any sensation of tackiness during or after their application.

There is also a need for cosmetic compositions that are stable over time and that are not impaired by variations in temperature, especially associated, for example, with changes in external/internal environment.

There is also a need for compositions that have a translucent and/or transparent appearance and that have improved gloss when applied.

The aim of the present invention is to satisfy all these needs.

Thus, according to one of its aspects, a subject of the present invention is a cosmetic composition for making up and/or caring for keratin materials, comprising:
at least one ester of polyol(s) and of fatty diacid dimer, or an ester thereof, with
at least one polyamide with an average molecular mass of less than 100000, comprising a) a polymer backbone with hydrocarbon-based repeating units containing at least one amide function, b) optionally a pendent fatty chain and/or at least one terminal fatty chain, which is optionally functionalized, containing from 6 to 120 carbon atoms, and being linked to these hydrocarbon-based units.

According to one embodiment, a composition according to the invention may also comprise fumed silica particles.

According to one embodiment, a composition according to the invention may have a hardness of less than or equal to about 30 g, or for example and in particular less than or equal to about 20 g, measured at about 20° C.

The inventors have observed, unexpectedly, that it is possible to satisfy all the abovementioned requirements with the proviso of combining in such a composition at least one polyamide and an ester of polyol(s) and of fatty diacid dimer, or an ester thereof.

According to one aspect, a composition in accordance with the invention may have the property of not flowing under its own weight.

These compositions may also be transparent and/or translucent and may give improved final gloss, especially to the lips.

According to one embodiment, on account of this translucency, the effect of the dyestuffs, for example of the nacres present in the compositions, is enhanced in the packaging.

A composition in accordance with the invention may be intended to be applied to keratin materials, and for example to bodily and/or facial skin, the lips or the integuments, such as the eyelashes.

According to one embodiment, the composition according to the invention may be a composition for the lips.

It may be in the form of a gloss.

It may also be in a compact form.

Thus, it may be applied by finger or using a suitable applicator, for instance a foam tip.

According to another of its aspects, a subject of the present invention is the use of at least one polyamide in accordance with the invention, as an agent for structuring an ester of polyol(s) and of fatty diacid dimer, or an ester thereof, for the preparation of a cosmetic composition. For example, this composition may have a hardness of less than about 30 g, measured at about 20° C.

According to another aspect, a subject of the present invention is a method for the preparation of a cosmetic composition comprising at least one step of structuring an ester of polyol(s) and of fatty diacid dimer, or an ester thereof, with at least one polyamide in accordance with the invention as a structuring agent.

According to another aspect, a subject of the present invention is also the use of a cosmetic composition in accordance with the invention to produce a glossy deposit.

According to yet another aspect, a subject. of the present invention is a process for making up and/or caring for keratin materials, and for example the skin and/or the lips, comprising at least one step that consists in applying to at least part of the said keratin materials a composition in accordance with the invention.

Other characteristics, aspects, subjects and advantages of the present invention will emerge even more clearly on reading the description and the examples that follow.

According to one aspect, the fatty phase of a composition according to the invention is gelled or structured.

For the purposes of the present invention, the term “gelled fatty phase” means a fatty phase in the form of a gel, i.e. a three-dimensional network that retains in its mesh a large amount of fatty phase.

For the purposes of the present patent application, the term “structured fatty phase” means a fatty phase in the form of a rigid gel.

A composition in accordance with the invention having a gelled or structured phase may have a hardness such that the composition does not flow under its own weight, at room temperature, and may for example have a hardness of less than or equal to about 30 g, measured at about 20° C.

Measurement of the Hardness

The hardness of a composition according to the invention may be determined by measuring the compression force, at 20° C., using a texturometer, for example sold under the name TA-XT2i by the company Rheo, equipped with a stainless steel cylinder 3 mm in diameter travelling at a measuring speed of 0.5 mm/s and penetrating into the cosmetic composition to a penetration depth of 2 mm.

For example, the measuring protocol may be as defined below.

A sample of cosmetic composition is heated to a temperature of 90-100° C. The molten sample is then poured into a container 4 mm deep and is then cooled to an ambient temperature of about 20-25° C. for 24 hours.

The sample is then stored for at least one hour at 20° C. before performing the hardness measurement.

The hardness value is the maximum compression force measured, divided by the area of the texturometer cylinder in contact with the sample of cosmetic composition.

A cosmetic composition in accordance with the invention may thus have a hardness ranging from 6 g to about 30 g, for example from about 10 g to about 25 g or for example of about 15 g, measured at about 20° C.

In parallel with this hardness, a composition according to the invention may be devoid of the capacity to flow under its own weight.

For the purposes of the present invention, the term “composition that does not flow under its own weight” means a composition having rheological behaviour at room temperature such that, after it has been heated to a temperature of 90-100° C. and then poured into a container, for example of cylindrical type, with a diameter of between 2 and 3 cm, and about 2-3 cm deep, and then cooled to an ambient temperature of about 20-25° C. for 24 hours, and when this container is inclined such that the axis passing through its orifice forms with the vertical axis an angle of 90°, for a period of about 6 hours, the composition, filling at least half the container, does not flow out of the container.

The composition according to the invention may have particular rheological behaviour, defined for example by a viscosity for a shear range defined and measured as follows.

The measurements may be performed using an RS 75 controlled-stress rheometer from the company ThermoRheo, equipped with a thermostatic bath and a stainless-steel spindle of cone/plate geometry, the cone having a diameter of 35 mm, an angle of 2° and a gap (distance between the lower plate—known as the stator plate—on which the composition is deposited, and the upper plate—known as the rotor plate) of 0.3 mm.

The measurements are performed at 25° C.±0.5° C.

The analysis in flow regime at equilibrium consists in subjecting a sample of composition, from a given moment, to an instantaneous shear stress τ, which is kept constant for a time t (waiting time chosen such that the permanent regime is reached, t=30 s).

Simultaneously, the change over time of the corresponding shear strain γ is monitored and the shear gradient \dot{γ} when equilibrium is reached is recorded.

In a first stage, the sample is maintained at a temperature of 25° C. for 2 minutes (without any applied shear).

Next, the flow at equilibrium is measured in controlled-stress mode.

Increasing stresses are applied to the sample starting from an initial stress equal to 0.089 Pa to arrive at a final stress of 3400 Pa, the stresses being applied once only.

Time is allowed for a stable value to be reached between each stress, the waiting time between each stress being 30 s.

The shear gradient range is from 10⁻⁴ s⁻¹ to 10³ s⁻¹. In the range under consideration, the maximum value of 10³ s⁻¹ should be taken into account with an uncertainty of measurement of ±150 s⁻¹.

Analysis of the results is performed by means of the graphic representation of the change in viscosity, written as η, as a function of the shear gradient, written as \dot{γ}.

The composition according to the invention may have a rheological profile such that, for a shear gradient range of from 10⁻² to 10 s⁻¹, the viscosity is greater than 10⁵ mPa.s.

The term “ambient temperature” above denotes the temperature of the environment in which a composition is placed and which may be set, for reproducible experimental conditions, between 20 and 25° C., but which may be varied, for example as a function of seasonal conditions and the nature of the environment.

Ester of Polyol(s) and of Fatty Diacid Dimer

In the expression “ester of polyol(s) and of fatty diacid dimer, or an ester thereof”, the term “or an ester thereof” means one of the derivatives of these esters and of polyol(s) and of fatty diacid dimer obtained either by reaction of alcohol function(s) of the polyol, not engaged in bonds of ester type with acid functions of the diacid dimer, with one or more carboxylic functions of acid molecules other than the diacid dimer, or alternatively by reaction of acid function(s) of the diacid dimer, not engaged in bonds of ester type with alcohol functions of the polyol, with alcohol functions of alcohol molecules other than the polyol.

Advantageously, the esters of polyol(s) and of fatty diacid dimer, or an ester thereof, which are suitable for use in the invention may have a viscosity, measured at about 25° C., of greater than or equal to about 1500 mPa.s.

The viscosity of an ester of polyol(s) and of fatty diacid dimer, or an ester thereof, according to the invention may be measured according to any process known to those skilled in the art, and for example according to the conventional process described hereinbelow.

The viscosity may be measured using a cone/plate or parallel plate viscometer of Ares type (TA-Instrument) operating in kinetic sweep mode over a shear range of about 1-1000 s⁻¹ to induce a flow tension of about 1000 Pa.

The cone/plate or parallel plates may consist of a material selected from the group consisting of stainless steel, acrylic resins or polyphenylene sulfide (PPS resin).

The cone/plate diameter may be 25 mm (cone angle 0.10 radiants).

The measurement is performed at about 25° C.

Before any measurement, the stability of the sample is checked by means of the dynamic sweep period test, which makes it possible to determine if the sample is stable per se.

The shear viscosity is determined using the ETA value in the plateau region according to the flow.

The dynamic sweep period is determined at a frequency of 1.0 Hz over a period of 600 seconds.

The measurements at constant sweep rate are performed with a rate ranging from 1.0 to 1000 s⁻¹ and for example from 1.0 to 100 s⁻¹.

The viscosity of an ester of polyol(s) and of fatty diacid dimer, or an ester thereof, suitable for use in the invention may range from about 1500 mPa.s to about 150 000 mPa.s, or for example from about 2000 mPa.s to about 150 000 mPa.s, or for example from about 15 000 mPa.s to about 100 000 mPa.s or for example from about 30 000 mPa.s to about 80 000 mPa.s.

According to one embodiment, a polyol that is suitable for use in the invention may be a diol dimer.

The esters of diol dimer and of fatty diacid dimer that may be used in the context of the present invention are commercially available or may be prepared in a conventional manner. They may be of plant origin and may be obtained by esterification of diacid dimers with diol dimers.

In an esterification reaction with a diacid dimer, a polyol dicarboxylate is obtained, which may have a weight-average molecular weight, determined by gel permeation chromatography (GPC), ranging from 2000 to 25 000 g/mol or for example between 2000 and 4000 g/mol.

Diacid Dimer

The fatty diacid dimers, or diacid dimers, may be conventionally obtained by polymerization reaction, for example by intermolecular dimerization, of at least one unsaturated fatty acid.

They may contain at least two carboxylic acid groups.

As indicated previously, the carboxylic functions of the fatty diacid dimer not engaged in the ester bond with the polyol residue(s) may be engaged in other ester bonds with other alcohol functions of alcohol molecules other than the polyol(s).

These alcohol molecules or residues may be monoalcohols or polyols.

As examples of alcohol residues that are suitable for use in the invention, mention may be made of hydrocarbon-based compounds comprising a hydroxyl function and containing from 4 to 40 carbon atoms, or for example from 6 to 36 carbon atoms, or for example from 8 to 32 carbon atoms, or for example from 16 to 28 carbon atoms, or for example from 18 to 24 carbon atoms.

As examples of monoalcohols that are suitable for the invention, mention may be made, in a non-limiting manner, of butanol, pentanol, propanol, hexanol, heptanol, octanol, decanol, dodecanol, hexadecanol, octadecanol, eicosadecanol, phytosterol, isostearol, stearol, cetol, behenol, etc.

The fatty diacid dimers may be derived for example from the dimerization of an unsaturated fatty acid, for example of C₈ to C₃₄, or for example of C₁₂ to C₂₂, in particular of C₁₆ to C₂₀ and for example of C₁₈.

As examples of these unsaturated fatty acids, mention may be made for example of undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid and arachidonic acid, and mixtures thereof.

According to one embodiment variant, it may be the diacid dimer from which the diol dimer to be esterified may also be derived. It may be its hydrogenated form.

The hydrogenated form of the diacid dimer may be partial or total, and may correspond, for example, to the saturated form, which is more stable towards oxidation.

According to another embodiment variant, it may be the diacid dimer derived from the dimerization of linoleic acid.

According to one embodiment, the diacid dimer, may be a commercial product, consisting of a dicarboxylic acid containing about 36 carbon atoms. This product may also contain a trimeric acid and a monomeric acid, in proportions that depend on the degree of purity of the product.

Products whose diacid dimer content may be greater than 70% and others whose diacid dimer content has been adjusted to 90% or more are conventionally found commercially.

Diacid dimers, and for example dilinoleic diacids whose stability towards oxidation has been improved by hydrogenation of the double bonds remaining after the dimerization reaction, may also be found commercially.

In the present invention, any diacid dimer that is currently commercially available may be used.

In an esterification reaction with a diacid dimer, the average degree of esterification and the average molecular weight of the ester obtained may be adjusted by varying the ratio of the diol dimer to the diacid dimer.

Polyols

The term “polyol” is intended to denote any hydrocarbon-based compound comprising at least two hydroxyl functions and containing from 4 to 40 carbon atoms, or for example from 6 to 36 carbon atoms, or for example from 8 to 32 carbon atoms, or for example from 16 to 28 carbon atoms and for example from 18 to 24 carbon atoms.

The hydrocarbon-based chains may be interrupted, where appropriate, by the presence of at least one heteroatom, and for example an oxygen atom.

A polyol or a polyol ester that is suitable for use in the present invention may comprise, for example, from 2 to 12 hydroxyl functions, or for example from 2 to 8 hydroxyl functions, or for example from 4 to 6 hydroxyl functions.

Where appropriate, the hydroxyl functions, other than those already employed in an ester bond with the diacid dimer, may also be employed, wholly or partly in other ester bonds via reaction with acid molecules other than the diacid dimer.

The polyol or an ester thereof that is suitable for use in the present invention may be selected from the group consisting of linear, branched, cyclic or polycyclic, saturated or unsaturated alcohols.

Thus, the polyol may be selected, for example, from the group consisting of a diol, a triol, a tetraol, or a pentaol, or an ester thereof.

The polyol may be a diol, or an ester thereof, selected for example from the group consisting of a fatty alcohol dimer, a monoglycerol or polyglycerol, a C₂₋₄ monoalkylene or polyalkylene glycol, 1,4-butanediol and pentaerythritol.

As examples of diols that are also suitable for use in the invention, mention may be made, in a non-exhaustive manner, of butanediol, pentanediol, propanediol, hexanediol, hexylene glycol, heptanediol, octanediol, nonanediol, decanediol, 1-decanediol, dodecanediol, tridecanediol, tetradecanediol, pentadecanediol, hexadecanediol, nonadecanediol, octadecanediol, cyclohexanediol, diglycerol, erythritol, pentaerythritol, xylitol, sorbitol, ethylene glycol and xylene glycol, and isomers thereof.

According to one variant, as an example of diols that are suitable for use the invention, mention may be made of diol dimers.

For the purposes of the present invention, the term “diol dimer” is intended to mean saturated diols derived from the hydrogenation of the corresponding diacid dimers, a diacid dimer being as defined above, and for example a diacid dimer of at least one unsaturated fatty acid.

As regards the industrially manufactured diol dimer, it also generally contains other components, for example a triol trimer, a monoalcohol and compounds of ether type, according to the degree of purification of the acid dimer and/or of the lower alcohol ester thereof, used as starting material.

Generally, products whose diol dimer content is greater than 70% may be used in the present invention. However, one may use a diol dimer of high purity, such as a compound whose diol dimer content is greater than 90%.

Thus, a diol dimer may be produced by hydrogenation, for example catalytic hydrogenation, of a diacid dimer, which is itself obtained by dimerization of at least one unsaturated fatty acid.

A suitable fatty acid may be such as those mentioned previously, and for example of C₈ to C₃₄, or for example of C₁₂ to C₂₂, or for example of C₁₆ to C₂₀ and more particularly of C₁₈.

According to one embodiment, the diol dimer may be derived from the hydrogenation of dilinoleic diacid.

A diol dimer may be, for example, dilinoleol.

The diol dimer may generally be in a saturated form.

As another example of a diol dimer that is suitable for use in the invention, mention may be made for example of diglycerol.

This compound is a glycerol dimer resulting from the condensation of two molecules of glycerol, with the loss of a water molecule.

The term “diglycerol” denotes any isomer combination that can result from such a condensation, for instance linear isomers, branched isomers and, where appropriate, cyclic isomers resulting from an intramolecular dehydration of a diglycerol molecule.

The diglycerol may be obtained via any process known to those skilled in the art and especially those described in patent EP 0 750 848.

As examples of acid molecules that can interact with one or more hydroxyl functions of the polyol, not engaged in the ester bond with the diacid dimer, mention may be made, in a non-limiting manner, of molecules derived from isostearic acid, behenic acid, phytosteric acid, stearic acid or cetylic acid.

An ester that is suitable for use in the present invention may be obtained by reacting a polyol or an ester thereof with a diacid dimer, and for example a dimerdilinoleic acid, in a molar ratio of about 1.0:0.2-1.0.

An ester that may be suitable for use in the present invention may be obtained by reacting a dimerdilinoleic acid with a dilinoleol and, where appropriate, at least one additional monoalcohol selected for example from the group consisting of behenol, isostearol, phytosterol, stearol and cetol, and mixtures thereof.

Thus, an ester used in the context of the present invention may be used in the form of a mixture of various esters, for example.

An ester that is suitable for use in the invention may be obtained, for example, by reacting a glycerol, an isostearic acid and a dimerdilinoleic acid, for example in a molar ratio of 1.0:0.2-1.0:0.5-0.9.

As examples of esters of dimerdilinoleic acid and of polyol(s), or an ester thereof, suitable for the invention, mention may be made of the esters described in patent applications JP 2003-226609, JP 2004-256515 and JP 2005-179377.

An ester of polyol(s) and of fatty diacid dimer, or an ester thereof, suitable for use in the present invention may have a molecular weight ranging from about 2000 to about 25 000 g/mol, for example from about 4000 to about 20 000 g/mol, for example from about 5000 to about 20 000 g/mol, for example from about 7000 to about 15 000 g/mol and for example from about 8000 to about 10 000 g/mol.

According to one embodiment, an ester in accordance with the invention may comprise an alternating sequence of diacid dimer residue(s) and of residue(s) related to the said polyol(s), and for example to the said diol(s), the said polyols or diols being, for example, as defined above.

Thus, in such a configuration, each of the two ends of the said sequence may bear, respectively, a unit OR′ and OR″ with R′ and R″ representing, independently of each other, a hydrogen atom or OR′ and OR″ representing, independently of each other, a C₂ to C₃₆, for example C₈ to C₂₄, for example C₁₂ to C₂₀ and for example C₁₆ to C₁₈ hydrocarbon based monoalcohol residue.

According to one embodiment, R′ and R″ may both represent a hydrogen atom.

According to one embodiment, OR′ and OR″ may both represent an identical or different hydrocarbon-based monoalcohol residue.

As examples of hydrocarbon-based monoalcohol residues OR′ and OR″ that may be suitable for the invention, mention may be made of fatty alcohol residues.

An ester that is suitable for use in the invention may be selected from the group consisting of esters of general formula (I), (II) or (IV) described below, or a mixture thereof.

According to one embodiment, an ester of polyol(s) and of fatty diacid dimer, or an ester thereof, that may be suitable for use in the present invention may have the general formula (I) below:
R₃—OCO—R₁(—COO—R₂—OCO—R₁)ₙ—COO—R₃ (I)
in which:
COR₁CO represents a fatty diacid dimer residue,
OR₂O represents a fatty alcohol dimer residue,
OR₃ represents a hydrocarbon-based monoalcohol residue, and
n is an integer ranging from 1 to 15, for example from 2 to 10 or for example from 5 to 7.

According to one embodiment variant, COR₁CO may represent a dimerdilinoleate residue.

According to one embodiment variant, OR₂O may represent a dimerdilinoleyl residue.

Moreover, OR₃ may represent a hydrocarbon-based monoalcohol residue selected, for example, from the group consisting of behenyl, isostearyl and phytosteryl residues, and mixtures thereof.

According to another embodiment, the ester of dimerdilinoleic acid and of polyol(s) and of fatty diacid dimer, or an ester thereof, that may be suitable for use in the invention may for example have the general formula (II) below: 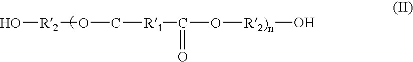
in which:
n is an integer ranging from 1 to 15, for example from 2 to 10 and in particular from 5 to 7
COR′₁CO represents a fatty diacid dimer residue,
OR′₂O represents a diglyceryl residue of general formula (III) below: 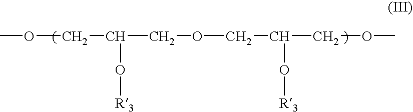
   in which:
R′₃ represents H or OR′₃ represents a fatty acid residue.

According to one embodiment variant, COR′₁CO may represent a dimerdilinoleate residue.

According to one embodiment variant, the fatty acid residue featured by OR′₃ may be an isostearyl residue.

According to one embodiment, an ester of dimerdilinoleic acid and of polyol(s) and of fatty diacid dimer, or an ester thereof, which may be suitable for use in the present invention, may be of formula (IV) below:
HO—R₁″—(—OCO—R₂″—COO—R₁″—)ₕ—OH (IV)
in which:

a) OR₁″O represents a diol dimer residue obtained by hydrogenation of a dimerdilinoleic acid,

b) COR₂″CO represents a fatty diacid dimer residue, and

c) h represents an integer ranging from 1 to 9, for example from 2 to 8 and for example from 4 to 6.

According to one embodiment, COR₂″CO may represent a dimerdilinoleate residue.

An ester that is suitable for the invention may be selected for example from group consisting of the esters having the following INCI nomenclature: polyglyceryl-2 isostearate dimerdilinoleate copolymer, bis-behenyl/isostearyl/phytosteryl dimerdilinoleyl dimerdilinoleate, dimerdilinoleyl dimerdilinoleate and mixtures thereof.

Such compounds may be obtained, for example, under the reference Hailuscent ISDA (Kokyu Alcohol) and Plandool-G, Lusplan DD-DA5, Lusplan DD-DA7, PHY/IS-DA and Lusplan DD-DAS (Nippon Fine Chemical Company Ltd).

For example, Lusplan DD-DA5 and Lusplan DD-DA7 are described in patent application FR 03/02809.

The ester of polyol(s) and of unsaturated fatty diacid dimer, or an ester thereof, is generally adjusted so as to control the gloss of the composition to a desired value.

In the present case, the ester may be present in a content ranging from about 1% to about 95% by weight, for example from about 5% to about 85% by weight, or for example from about 10% to about 60% by weight relative to the total weight of the composition.

Polyamide

A composition according to the present invention comprises at least one polymer with a weight-average molecular mass of less than 100 000, or for example less than 50 000, comprising a) a polymer backbone with hydrocarbon-based repeating units containing at least one amide function, and b) optionally at least one pendent fatty chain and/or at least one terminal fatty chain, which are optionally functionalized, containing from 6 to 120 carbon atoms and especially from 8 to 120 carbon atoms, and being linked to these hydrocarbon-based units.

For the purposes of the invention, the term “functionalized chains” means an alkyl chain comprising one or more functional or reactive groups selected for example from the group consisting of amide, hydroxyl, ether, oxyalkylene or polyoxyalkylene, halogen including fluoro or perfluoro groups, ester, siloxane and polysiloxane groups. In addition, the hydrogen atoms of one or more fatty chains may be at least partially substituted with fluorine atoms.

According to the invention, these chains may be directly linked to the polymer backbone or via an ester function or a perfluoro group.

For the purposes of the invention, the term “polymer” means a compound containing at least two repeating units and for example at least three repeating units.

For the purposes of the invention, the term “hydrocarbon-based repeating unit” means a unit containing from 2 to 80 carbon atoms and for example from 2 to 60 carbon atoms, bearing hydrogen atoms and possibly oxygen atoms, which may be linear, branched or cyclic, and saturated or unsaturated. These units each also comprise one or more amide functions that may be non-pendent and that may be within the polymer backbone.

According to one embodiment, pendent fatty chains may be linked directly to at least one of the nitrogen atoms of the amide units.

A polyamide that is suitable for use in the invention may comprise, between the hydrocarbon-based units, silicone units or oxyalkylene units.

In addition, the fatty chains of a polyamide that is suitable for use in the composition of the invention may contain from 40% to 98% of the total number of amide units and of fatty chains and in particular from 50% to 95% of the total number of amide units and of fatty chains.

A polyamide according to the invention may have a number-average molecular mass of less than 100 000, for example ranging from 1000 to 100 000, or for example ranging from 1000 to 50 000, or for example ranging from 1000 to 30 000, or for example ranging from 2000 to 20 000 or for example ranging from 2000 to 10 000 g/mol.

A polyamide that is suitable for use in the invention may be insoluble in water, especially at 25° C.According to one embodiment, it may contain no ionic groups.

As examples of polyamides that may be used in the invention, mention may be made of polyamides branched with pendent fatty chains and/or terminal fatty chains containing from 6 to 120 carbon atoms or for example from 8 to 120 or for example from 12 to 68 carbon atoms.

According to one embodiment, the terminal fatty chain(s) may be linked to the polyamide backbone via bonding groups, for example ester groups.

According to one embodiment, a polyamide that is suitable for use in the invention may comprise a fatty chain at each end of the polymer backbone. Other suitable bonding groups that may be mentioned include ether, amine, urea, urethane, thioester, thiourea and thiourethane groups.

A polyamide that is suitable for use in the invention may be a polyamide resulting from a polycondensation of a dicarboxylic acid containing at least 32 carbon atoms (for example containing from 32 to 44 carbon atoms) with an amine selected from the group consisting of diamines containing at least 2 carbon atoms (for example from 2 to 36 carbon atoms) and triamines containing at least 2 carbon atoms (for example from 2 to 36 carbon atoms).

A diacid that is suitable for use in the invention may be, for example, a dimer derived from an ethylenically unsaturated fatty acid containing at least 16 carbon atoms or for example from 16 to 24 carbon atoms, for instance oleic acid, linoleic acid or linolenic acid.

Examples of diamines that may be mentioned include ethylenediamine, hexylenediamine and hexamethylenediamine. The triamine may be, for example, ethylenetriamine.

For the polymers comprising one or two terminal carboxylic acid groups, it may be possible to esterify them with a monoalcohol containing at least 4 carbon atoms, for example from 10 to 36 carbon atoms, or for example from 12 to 24 carbon atoms, or for example from 16 to 24 carbon atoms, for example 18 carbon atoms.

These polymers may be, for example, those described in document U.S. Pat. No. 5,783,657 from the company Union Camp.

According to one embodiment variant, the polyamide may be of general formula (V) below: 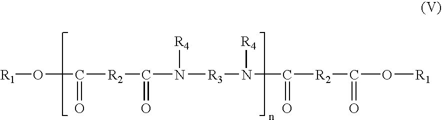
in which:
n may denote a whole number of amide units such that the number of ester groups represents from 10% to 50% of the total number of ester and amide groups;
each of the symbols R₁ may independently denote an alkyl or alkenyl group containing at least 4 carbon atoms and for example from 4 to 24 carbon atoms;
each of the symbols R₂ may independently represent a C₄ to C₄₂ hydrocarbon-based group, on condition that 50% of the groups R₂ represent a C₃₀ to C₄₂ hydrocarbon-based group;
each of the symbols R₃ may represent independently at each instance an organic group containing at least two carbon atoms, hydrogen atoms and optionally one or more oxygen or nitrogen atoms;
and each of the symbols R4 may independently represent a hydrogen atom, a C₁-C₁₀ alkyl group or a direct bond to R₃ or to another R₄ such that the nitrogen atom to which R₃ and R₄ are both bonded forms part of a heterocyclic structure defined by R₄—N—R₃, with at least 50% of the R₄ representing a hydrogen atom.

In the case of formula (I), the optionally functionalized terminal fatty chains for the purposes of the invention may be terminal chains linked to the last nitrogen atom of the polyamide backbone.

In one variant, the ester groups of formula (I), which form part of the terminal and/or pendent fatty chains for the purposes of the invention, may represent from 15% to 40%, or for example from 20% to 35% of the total number of ester and amide groups.

Furthermore, n may represent an integer ranging from 1 to 5, or for example greater than 2.

R₁ may be a C₁₂-C₂₂, or for example C₁₆-C₂₂ alkyl group.

R₂ may be a C₁₀-C₄₂ hydrocarbon-based (alkylene) group, and for example at least 50%, or for example at least 75% of the R₂ may be hydrocarbon-based groups containing from 30 to 42 carbon atoms. The other R₂ may be C₄-C₁₉ or for example C₈-C₁₂ hydrocarbon-based groups.

R₃ may represent a C₂-C₃₆ hydrocarbon-based group or a polyoxyalkylene group.

In one variant, R₃ may represent a C₄-C₁₂ hydrocarbon-based group.

R₄ may represent a hydrogen atom.

The hydrocarbon-based groups may be linear, cyclic or branched, and saturated or unsaturated. Moreover, the alkyl and alkylene groups may be linear or branched, and saturated or unsaturated.

The polymers of formula (I) may be in the form of mixtures of polymers. These mixtures may also contain a synthetic product corresponding to a compound of formula (I) in which n is 0, i.e. a diester.

According to one embodiment, a polyamide that is suitable for use in the invention may be selected from the group consisting of C₃₆ diacid copolymers condensed onto an ethylenediamine with a weight-average molecular mass of about 6000, and mixtures thereof.

As examples of polymers that may be used in the compositions according to the invention, mention may be made of the commercial products sold by the company Arizona Chemical under the names Uniclear 80 and Uniclear 100. They are sold, respectively, in the form of a gel at 80% (active material) in a mineral oil and at 100% (active material). They have a softening point of from 88 to 94° C. These commercial products are a mixture of copolymers of a C₃₆ diacid condensed onto ethylenediamine, with a weight-average molecular mass of about 6000. The terminal ester groups result from the esterification of the remaining acid end groups with cetyl alcohol, stearyl alcohol or mixtures thereof (also known as cetylstearyl alcohol).

As polyamides that may be used in the compositions according to the invention, mention may also be made of polyamide resins resulting from the condensation of an aliphatic dicarboxylic acid and of a diamine (including compounds containing more than two carbonyl groups and two amine groups), the carbonyl and amine groups of adjacent individual units being condensed via an amide bond. These polyamide resins are for example those sold under the brand name Versamid® by the companies General Mills Inc. and Henkel Corp. (Versamid 930, 744 or 1655) or by the company Olin Mathieson Chemical Corp., under the brand name Onamid®, especially Onamid S or C. These resins have a weight-average molecular mass ranging from 6000 to 9000. For further information regarding these polyamides, reference may be made to documents U.S. Pat. No. 3,645,705 and U.S. Pat. No. 3,148,125. Versamid® 930 or 744 is for example used.

It is also possible to use the polyamides sold by the company Arizona Chemical under the reference Uni-Rez (2658, 2931, 2970, 2621, 2613, 2624, 2665, 1554, 2623, 2662) and the product sold under the reference Macromelt 6212 by the company Henkel. For further information regarding these polyamides, reference may be made to document U.S. Pat. No. 5,500,209.

It is also possible to use polyamide resins derived from vegetables, such as those described in patents U.S. Pat. No. 5,783,657 and U.S. Pat. No. 5,998,570.

A polyamide that is suitable for preparing a composition according to the invention may have a softening point of greater than 65° C., which may be up to 190° C. For example, it may have a softening point ranging from 70 to 130° C. and for example from 80 to 105° C.

In general, the polyamide particles are present in a composition according to the invention in an amount sufficient to adjust the hardness of the said composition to the required value.

A polyamide may be present in a composition according to the invention in a content ranging from about 0.01% to about 20% by weight, for example from about 0.1% to about 10% by weight, or for example from about 0.2% to about 4% by weight relative to the total weight of the composition.

Fumed Silica

The composition according to the invention may also comprise fumed silica particles.

The fumed silica particles that are suitable for use in the invention may be hydrophilic or surface-treated in order to be made hydrophobic.

The hydrophilic fumed silicas may be obtained by high-temperature hydrolysis of a volatile silicon compound in an oxyhydric flame, producing a finely divided silica. The hydrophilic silicas may have a large number of silanol groups at their surface.

Such hydrophilic silicas are sold, for example, under the names Aerosil 130®, Aerosil 200®, Aerosil 255®, Aerosil 300® and Aerosil 380® by the company Degussa, and Cab-O-Sil HS-5®, Cab-O-Sil EH-5®, Cab-O-Sil LM-130®, Cab-O-Sil MS-55® and Cab-O-Sil M-5® by the company Cabot.

The hydrophobic fumed silicas may be obtained by modifying the surface of the silica via a chemical reaction that generates a reduction in the number of silanol groups, these groups possibly being substituted for example with hydrophobic groups.

The hydrophobic groups may be:
trimethylsiloxyl groups, which are for example obtained by treating fumed silica in the presence of hexamethyldisilazane. Silicas thus treated are known as “Silica silylate” according to the CTFA (6th edition, 1995). They are sold, for example, under the references Aerosil R812® by the company Degussa and Cab-O-Sil TS-530® by the company Cabot,
dimethylsilyloxyl or polydimethylsiloxane groups, which are for example obtained by treating fumed silica in the presence of polydimethylsiloxane or dimethyldichlorosilane. Silicas thus treated are known as “Silica dimethyl silylate” according to the CTFA (6th edition, 1995). They are sold, for example, under the references Aerosil R972 and Aerosil R974 by the company Degussa, and Cab-O-Sil TS-610® and Cab-O-Sil TS-720® by the company Cabot.

Fumed silica particles may be present in a composition in accordance with the invention in a content ranging from about 1% to about 30% by weight, for example from about 5% to about 20% by weight, or for example from about 10% to about 15% by weight relative to the total weight of the composition.

According to one embodiment, the content of all of the compounds included in a composition in accordance with the invention, i.e. at least one ester of polyol(s) and of fatty diacid dimer, or an ester thereof, and at least one polyamide, may be adjusted to 100%.

Other compounds usually used in cosmetics, such as liquid or solid fatty phases, cosmetic active agents, fillers or dyestuffs, may be additionally present in amounts adjusted such that the properties of a composition in accordance with the invention, especially in terms of hardness, gloss and/or transparency, are not thereby substantially impaired.

Physiologically Acceptable Medium

A composition in accordance with the invention comprises a physiologically acceptable medium.

The term “physiologically acceptable medium” denotes a medium that may be suitable for applying a composition according to the invention to the skin and/or the lips. The physiologically acceptable medium is generally suited to the nature of the support onto which the composition is to be applied, and also to the aspect in which the composition is intended to be packaged.

The physiologically acceptable medium may comprise an aqueous phase essentially comprising water.

It may also comprise a mixture of water and of water-miscible solvent (miscibility in water of greater than 50% by weight at 25° C.), for instance one or a mixture of lower monoalcohols(s) containing from 1 to 5 carbon atoms, such as ethanol or isopropanol, glycols containing from 2 to 8 carbon atoms, such as propylene glycol, ethylene glycol, 1,3-butylene glycol or dipropylene glycol, C₃-C₄ ketones and C₂-C₄ aldehydes, and mixtures thereof.

According to another embodiment, the composition in accordance with the invention may be anhydrous.

The term “anhydrous composition” means a composition comprising less than 10% by weight, for example less than 5% by weight, for example less than 3% by weight, for example less than 2% by weight, or for example less than 1% by weight of water relative to the total weight of the composition.

Fatty Phase

A cosmetic composition in accordance with the present invention may comprise, besides the ester of polyol(s) and of fatty diacid dimer, an additional fatty phase, for example a liquid fatty phase, comprising one or more oils, and/or a fatty phase that is solid at a temperature of about 20-25° C. and at atmospheric pressure, and mixtures thereof.

The term “oil” means any fatty substance that is in liquid form at a temperature of about 20-25° C. and at atmospheric pressure. The liquid fatty phase may also contain, in addition to oils, other compounds dissolved in the oils, such as gelling agents and/or structuring agents.

The oil(s) may be present in a proportion of from 0.1% to 99% by weight, for example from at least 1% to 90% by weight, for example from 5% to 70% by weight, for example from 10% to 60% by weight or for example from 20% to 50% by weight relative to the total weight of the cosmetic composition according to the invention.

The liquid fatty phase that may be suitable for preparing a cosmetic composition according to the invention may be selected from the group consisting of volatile or non-volatile, silicone or non-silicone oils, and mixtures thereof.

For the purposes of the present invention, the term “silicone oil” means an oil comprising at least one silicon atom, and for example at least one Si—O group.

The term “hydrocarbon-based oil” means an oil mainly containing hydrogen and carbon atoms, and possibly oxygen, nitrogen, sulfur and/or phosphorus atoms.

Volatile Oils

A composition in accordance with the invention may comprise at least one volatile oil.

For the purposes of the present invention, the term “volatile oil” means an oil (or non-aqueous medium) capable of evaporating on contact with the skin in less than one hour, at room temperature and at atmospheric pressure. The volatile oil is a volatile cosmetic oil, which is liquid at room temperature, for example having a non-zero vapour pressure, at room temperature and atmospheric pressure, and for example having a vapour pressure ranging from 0.13 Pa to 40 000 Pa (10⁻³ to 300 mmHg), particularly ranging from 1.3 Pa to 13 000 Pa (0.01 to 100 mmHg), or for example ranging from 1.3 Pa to 1300 Pa (0.01 to 10 mmHg).

The volatile hydrocarbon-based oils may be selected from the group consisting of hydrocarbon-based oils containing from 8 to 16 carbon atoms, and for example branched C₈-C₁₆ alkanes (also known as isoparaffins), for instance isododecane (also known as 2,2,4,4,6-pentamethylheptane), isodecane, isohexadecane and, for example, the oils sold under the trade names Isopar® or Permethyl®.

Volatile oils that may also be used include volatile silicones, for instance volatile linear or cyclic silicone oils, especially those with a viscosity ≦8 centistokes (8×10⁻⁶ m²/s), and containing from 2 to 10 silicon atoms, for example from 2 to 7 silicon atoms, these silicones optionally comprising alkyl or alkoxy groups containing from 1 to 10 carbon atoms. As a volatile silicone oil that may be used in the invention, mention may be made for example of dimethicones with a viscosity of 5 and 6 cSt, octamethylcyclotetrasiloxane, decamethylcyclopentasiloxane, dodecamethylcyclo-hexasiloxane, heptamethylhexyltrisiloxane, heptamethyloctyltrisiloxane, hexam ethyldi-siloxane, octamethyltrisiloxane, decamethyltetrasiloxane and dodecamethylpentasiloxane, and mixtures thereof.

Volatile fluoro oils such as nonafluoromethoxybutane or perfluoromethyl-cyclopentane, and mixtures thereof, may also be used.

It is also possible to use a mixture of the oils mentioned above.

A cosmetic composition according to the invention may comprise at least one volatile oil in a content ranging from about 1% to about 50% by weight, for example ranging from about 2% to about 40% by weight, or for example ranging from about 5% to about 30% by weight of volatile oil relative to the total weight of the composition.

Non-volatile Oils For the purposes of the present invention, the term “non-volatile oil” means an oil with a vapour pressure of less than 0.13 Pa and for example oils of high molar mass. The non-volatile oils may be hydrocarbon-based oils for example of animal origin or of plant origin, oils of synthetic or mineral origin, silicone oils or fluoro oils, or mixtures thereof.

A cosmetic composition according to the present invention may also comprise at least one non-volatile oil.

The non-volatile oils may be selected for example from the group consisting of non-volatile hydrocarbon-based oils, which may be fluorinated, and/or non-volatile silicone oils.

As non-volatile hydrocarbon-based oils that are suitable for use in the invention, mention may be made for example of:
hydrocarbon-based oils of animal origin,
hydrocarbon-based oils of plant origin such as phytostearyl esters, such as phytostearyl oleate, phytostearyl isostearate and lauroyl/octyldodecyl/phytostearyl glutamate, for example sold under the name Eldew PS203 by Ajinomoto, triglycerides consisting of fatty acid esters of glycerol, the fatty acids of which may have chain lengths ranging from C₄ to C₂₄, these chains possibly being linear or branched, and saturated or unsaturated; these oils are for example heptanoic or octanoic triglycerides, wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cotton seed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion flower oil or musk rose oil; shea butter; or alternatively caprylic/capric acid triglycerides, for instance those sold by the company Stearineries Dubois or those sold under the names Miglyol 810®, 812® and 818® by the company Dynamit Nobel,
hydrocarbon-based oils of mineral or synthetic origin, for instance:
   synthetic ethers containing from 10 to 40 carbon atoms;
   linear or branched hydrocarbons of mineral or synthetic origin such as petroleum jelly, polydecenes, hydrogenated polyisobutene such as Parleam, and squalane, and mixtures thereof, and for instance hydrogenated polyisobutene,
   synthetic esters, for instance oils of formula R₁COOR₂ in which R₁ represents a linear or branched fatty acid residue containing from 1 to 40 carbon atoms and R₂ represents a hydrocarbon-based chain that is for example branched, containing from 1 to 40 carbon atoms provided that R₁+R₂≧10.

The esters may be selected for example from the group consisting of fatty acid esters, for example:
cetostearyl octanoate, isopropyl alcohol esters, such as isopropyl myristate or isopropyl palmitate, ethyl palmitate, 2-ethylhexyl palmitate, isopropyl stearate or isostearate, isostearyl isostearate, octyl stearate, hydroxylated esters, for instance isostearyl lactate, octyl hydroxystearate, diisopropyl adipate, heptanoates, and especially isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate and palmitate, alkyl benzoate, polyethylene glycol diheptanoate, propylene glycol 2-diethylhexanoate, and mixtures thereof, C₁₂ to C₁₅ alcohol benzoates, hexyl laurate, neopentanoic acid esters, for instance isodecyl neopentanoate, isotridecyl neopentanoate, isostearyl neopentanoate and octyldodecyl neopentanoate, isononanoic acid esters, for instance isononyl isononanoate, isotridecyl isononanoate and octyl isononanoate, and hydroxylated esters, for instance isostearyl lactate and diisostearyl malate;
polyol esters and pentaerythritol esters, for instance dipentaerythrityl tetrahydroxystearate/tetraisostearate;
fatty alcohols that are liquid at room temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol and 2-undecylpentadecanol;
higher fatty acids such as oleic acid, linoleic acid or linolenic acid, and mixtures thereof; and
dialkyl carbonates, the two alkyl chains possibly being identical or different, such as dicaprylyl carbonate sold under the name Cetiol CC® by Cognis;
non-volatile silicone oils, for instance non-volatile polydimethylsiloxanes (PDMS), polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenylmethyldiphenyl-trisiloxanes and 2-phenylethyl trimethylsiloxysilicates, and dimethicones or phenyltrimethicones with a viscosity of less than or equal to 100 cSt, and mixtures thereof;
and mixtures thereof.

The non-volatile oils may be present in a composition according to the invention in a content ranging from 5% to 90% by weight, for example from 25% to 80% by weight and for example from 40% to 70% by weight relative to the total weight of the composition.

Other Fatty Substances

A composition according to the invention may also comprise at least one fatty substance selected from the group consisting of waxes and pasty fatty substances, and mixtures thereof.

The wax is solid at room temperature (25° C.), with a reversible solid/liquid change of state, having a melting point of greater than 30° C., which may be up to 200° C., a hardness of greater than 0.5 MPa and having in the solid state an anisotropic crystal organization.

It may be a hydrocarbon-based wax, a fluoro wax and/or a silicone wax and may be of animal, plant, mineral or synthetic origin.

It may beselected, for example, from the group consisting of beeswax, carnauba wax, candelilla wax, paraffin waxes, hydrogenated castor oil, synthetic waxes, for instance polyethylene waxes (preferably with a molecular weight of between 400 and 600) or Fischer-Tropsch waxes, silicone waxes, for instance alkyl or alkoxy dimethicones containing from 16 to 45 carbon atoms, ceresins or ozokerites, for instance isoparaffins with a melting point of less than 40° C., such as EMW-0003 sold by the company Nippon Seirou, α-olefin oligomers, such as the polymers Performa V® 825, 103 and 260 sold by the company New Phase Technologies; ethylene-propylene copolymers, such as Performalene® EP 700, and microcrystalline waxes with a melting point of greater than 85° C., such as Hi-Mic® 1070, 1080, 1090 and 3080, sold by Nippon Seirou, and mixtures thereof.

According to one embodiment, the content of wax(es) used in a cosmetic composition in accordance with the invention may be less than or equal to about 5% by weight relative to the total weight of the composition.

According to another embodiment, a composition in accordance with the invention may be free of wax.

A cosmetic composition in accordance with the present invention may also comprise at least one pasty compound.

For the purposes of the present invention, the term “pasty compound” means a fatty compound with a reversible solid/liquid change of state, and comprising at a temperature of 23° C. a liquid fraction and a solid fraction. The term “pasty substance” also means polyvinyl laurate.

For the purposes of the invention, a pasty compound may have a hardness at 20° C. ranging from 0.001 to 0.5 MPa and for example from 0.002 to 0.4 MPa.

Among the pasty compounds that may be used in the composition according to the invention, mention may be made of lanolins and lanolin derivatives, for instance acetylated lanolins, oxypropylenated lanolins or isopropyl lanolate, and mixtures thereof. Esters of fatty acids or of fatty alcohols may also be used, for instance those containing from 20 to 65 carbon atoms, for instance triisostearyl or cetyl citrate; arachidyl propionate; polyvinyl laurate; cholesterol esters, for instance triglycerides of plant origin such as hydrogenated plant oils, viscous polyesters and mixtures thereof. Triglycerides of plant origin that may be used include hydrogenated castor oil derivatives, such as Thixinr® from Rheox.

Mention may also be made of polyesters resulting from the esterification of a carboxylic acid and of an aliphatic hydroxycarboxylic acid ester. For example, Risocast® DA-L (ester derived from the esterification reaction of hydrogenated castor oil with dilinoleic acid in proportions of 2 to 1) and Risocast® DA-H (ester resulting from the esterification of hydrogenated castor oil with isostearic acid in proportions of 4 to 3) sold by the Japanese company Kokyu Alcohol Kogyo.

As pasty compounds that may be suitable for formulating the cosmetic compositions in accordance with the present invention, mention may be made of hydrogenated cocoglycerides.

Mention may also be made of pasty silicone compounds such as high molecular weight polydimethylsiloxanes (PDMS) and for instance those containing pendent chains of the alkyl or alkoxy type containing from 8 to 24 carbon atoms, and having a melting point of 20-55° C., for instance stearyl dimethicones, for example those sold by the company Dow Corning under the trade names DC2503® and DC25514®, and mixtures thereof.

Dyestuff

A cosmetic composition in accordance with the invention may also comprise at least one dyestuff.

According to one embodiment, one or more dyestuffs may be added to a cosmetic composition according to the invention such that its transparency and/or translucency is not affected.

Such a dyestuff may be selected, for example, from the group consisting of water-soluble or water-insoluble, liposoluble or non-liposoluble, organic or mineral dyestuffs, for instance of pigment or nacre type, conventionally used in cosmetic compositions, and materials with an optical effect, and mixtures thereof.

The dyestuffs may be present in a proportion of from 0.01% to 40% by weight and for example from 0.5% to 25% by weight relative to the total weight of the cosmetic composition.

The term “pigments” should be understood as meaning white or coloured, inorganic (mineral) or organic particles that are insoluble in an aqueous solution, which are intended to colour and/or opacify the resulting film.

As mineral pigments that may be used in the invention, mention may be made of titanium oxide, zirconium oxide or cerium oxide, and also zinc oxide, iron oxide or chromium oxide, ferric blue, manganese violet, ultramarine blue and chromium hydrate.

It may also be a pigment having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30.

The dyestuff may also comprise a pigment having a structure that may be, for example, of silica microsphere type containing iron oxide. An example of a pigment having this structure is the product sold by the company Miyoshi under the reference PC BALL PC-LL-100 P, this pigment consisting of silica microspheres containing yellow iron oxide.

Among the organic pigments that may be used in the invention, mention may be made of carbon black, pigments of D&C type, lakes based on cochineal carmine or on barium, strontium, calcium or aluminium, or alternatively the diketopyrrolopyrroles (DPP) described in documents EP-A-542669, EP-A-787730, EP-A-787731 and WO-A-96/08537.

The term “nacres” should be understood as meaning coloured particles of any form, which may or may not be iridescent, for instance produced by certain molluscs in their shell, or alternatively synthesized, and which have a colour effect via optical interference.

The nacres may be selected from the group consisting of nacreous pigments such as titanium mica coated with an iron oxide, mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic dyestuffs.

Examples of nacres that may also be mentioned include natural mica coated with titanium oxide, with iron oxide, with natural pigment or with bismuth oxychloride.

Among the commercially available nacres that may be mentioned are the nacres Timica, Flamenco and Duochrome (on mica base) sold by the company Engelhard, the Timiron nacres sold by the company Merck, the Prestige nacres on mica base sold by the company Eckart and the Sunshine nacres on synthetic mica base sold by the company Sun Chemical.

The nacres may more particularly have a yellow, pink, red, bronze, orange, brown and/or coppery colour or tint.

As illustrations of nacres that may be used in the context of the present invention, mention may be made in particular of gold-coloured nacres sold for instance by the company Engelhard under the name Brilliant gold 212G (Timica), Gold 222C (Cloisonne), Sparkle gold (Timica), Gold 4504 (Chromalite) and Monarch gold 233X (Cloisonne); the bronze nacres sold for instance by the company Merck under the names Bronze fine (17384) (Colorona) and Bronze (17353) (Colorona) and by the company Engelhard under the name Super bronze (Cloisonne); the orange nacres sold for instance by the company Engelhard under the names Orange 363C (Cloisonne) and Orange MCR 101 (Cosmica) and by the company Merck under the names Passion orange (Colorona) and Matte orange (17449) (Microna); the brown-tinted nacres sold for instance by the company Engelhard under the names Nu-antique copper 340XB (Cloisonne) and Brown CL4509 (Chromalite); the nacres with a copper tint sold for instance by the company Engelhard under the name Copper 340A (Timica); the nacres with a red tint sold for instance by the company Merck under the name Sienna fine (17386) (Colorona); the nacres with a yellow tint sold for instance by the company Engelhard under the name Yellow (4502) (Chromalite); the red-tinted nacres with a golden tint sold for instance by the company Engelhard under the name Sunstone G012 (Gemtone); the pink nacres sold for instance by the company Engelhard under the name Tan opale G005 (Gemtone); the black nacres with a golden tint sold for instance by the company Engelhard under the name Nu antique bronze 240 AB (Timica); the blue nacres sold for instance by the company Merck under the name Matte blue (17433) (Microna); the white nacres with a silvery tint sold for instance by the company Merck under the name Xirona Silver; and the golden-green pinkish-orange nacres sold for instance by the company Merck under the name Indian summer (Xirona), and mixtures thereof.

The cosmetic composition according to the invention may also contain at least one material with a specific optical effect.

This effect is different from a simple conventional hue effect, i.e. a unified and stabilized effect as produced by standard dyestuffs, for instance monochromatic pigments.

For the purposes of the invention, the term “stabilized” means lacking the effect of variability of the colour with the angle of observation or in response to a temperature change.

For example, this material may be selected from the group consisting of particles with a metallic tint, goniochromatic colouring agents, diffracting pigments, thermochromic agents, optical brighteners, and also fibres, for instance interference fibres. Needless to say, these various materials may be combined so as to afford the simultaneous manifestation of two effects, or even of a novel effect in accordance with the invention.

The particles with a metallic tint that may be used in the invention may be selected from:
particles of at least one metal and/or of at least one metal derivative,
particles comprising a monomaterial or multimaterial organic or mineral substrate, at least partially coated with at least one layer with a metallic tint comprising at least one metal and/or at least one metal derivative, and
mixtures of the said particles.

Among the metals that may be present in the said particles, mention may be made, for example, of Ag, Au, Cu, Al, Ni, Sn, Mg, Cr, Mo, Ti, Zr, Pt, Va, Rb, W, Zn, Ge, Te and Se and mixtures or alloys thereof. Ag, Au, Cu, Al, Zn, Ni, Mo and Cr, and mixtures or alloys thereof (for example bronzes and brasses) are suitable metals.

The term “metal derivatives” is intended to denote compounds derived from metals, for instance oxides, fluorides, chlorides and sulfides.

Illustrations of these particles that may be mentioned include aluminium particles, such as those sold under the names Starbrite 1200 EAC® by the company Siberline and Metalure® by the company Eckart.

Mention may also be made of metal powders of copper or of alloy mixtures such as the references 2844 sold by the company Radium Bronze, metallic pigments, for instance aluminium or bronze, such as those sold under the names Rotosafe 700 from the company Eckart, silica-coated aluminium particles sold under the name Visionaire Bright Silver from the company Eckart, and metal alloy particles, for instance the silica-coated bronze (alloy of copper and zinc) powders sold under the name Visionaire Bright Natural Gold from the company Eckart.

They may also be particles comprising a glass substrate, for instance those sold by the company Nippon Sheet Glass under the name Microglass Metashine.

The goniochromatic colouring agent may beselected, for example, from the group consisting of interference multilayer structures and liquid-crystal colouring agents.

Examples of symmetrical interference multilayer structures that may be used in compositions produced in accordance with the invention are, for example, the following structures: Al/SiO₂/Al/SiO₂/Al, pigments having this structure being sold by the company Dupont de Nemours; Cr/MgF₂/Al/MgF₂/Cr, pigments having this structure being sold under the name Chromaflair by the company Flex; MoS₂/SiO₂/Al/SiO₂/MoS₂; Fe₂O₃/SiO₂/Al/SiO₂/Fe₂O₃ and Fe₂O₃/SiO₂/Fe₂O₃/SiO₂/Fe₂O₃, pigments having these structures being sold under the name Sicopearl by the company BASF; MoS₂/SiO₂/mica oxide/SiO₂/MoS₂; Fe₂O₃/SiO₂/mica oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona by the company Merck (Darmstadt). By way of example, these pigments may be the pigments of silica/titanium oxide/tin oxide structure sold under the name Xirona Magic by the company Merck, the pigments of silica/brown iron oxide structure sold under the name Xirona Indian Summer by the company Merck and the pigments of silica/titanium oxide/mica/tin oxide structure sold under the name Xirona Caribbean Blue by the company Merck. Mention may also be made of Infinite Colors pigments from the company Shiseido. Depending on the thickness and the nature of the various coats, different effects are obtained. Thus, with the structure Fe₂O₃/SiO₂/Al/SiO₂/Fe₂O₃, the colour changes from green-golden to red-grey for SiO₂ layers of from 320 to 350 nm; from red to golden for SiO₂ layers of from 380 to 400 nm; from violet to green for SiO₂ layers of from 410 to 420 nm; from copper to red for SiO₂ layers of from 430 to 440 nm.

Examples of pigments with a polymeric multilayer structure that may be mentioned include those sold by the company 3M under the name Color Glitter.

Examples of liquid-crystal goniochromatic particles that may be used include those sold by the company Chenix and also the product sold under the name Helicone® HC by the company Wacker.

The liposoluble dyes are, for example, Sudan Red, DC Red 17, DC Green 6, β-carotene, soybean oil, Sudan Brown, DC Yellow 11, DC Violet 2, DC Orange 5 and quinoline yellow.

The water-soluble dyes are, for example, beetroot juice or methylene blue.

According to one embodiment, a composition according to the invention may comprise at least one dyestuff chosen, for example, from organic dyestuffs and inorganic dyestuffs, such as pigments and nacres, and materials with a specific optical effect, and mixtures thereof.

According to another embodiment, the composition according to the invention is free of dyestuff.

Fillers

The cosmetic compositions in accordance with the invention may also comprise at least one filler, of organic or mineral nature.

For the purposes of the invention, the fillers are distinct of dyestuffs.

One or more fillers may be added to a cosmetic composition according to the invention such that these properties, for instance of viscosity, transparency and translucency, are not thereby substantially affected.

The term “filler” should be understood as meaning colourless or white solid particles of any form, which are in an insoluble form and dispersed in the medium of the composition. Being of mineral or organic nature they make it possible to impart body or rigidity to the composition, and/or softness, a matt effect and uniformity to the makeup. They are different from the fumed silica particles.

The fillers used in the compositions according to the present invention may be of lamellar, globular or spherical form, fibres or in any other form intermediate between these defined forms.

The fillers according to the invention may or may not be surface-coated, and in particular they may be surface-treated with silicones, amino acids, fluoro derivatives or any other substance that promotes the dispersion and compatibility of the filler in the composition.

Among the mineral fillers that may be used in the compositions according to the invention, mention may be made of talc, mica, silica, trimethyl siloxysilicate, kaolin, bentone, precipitated calcium carbonate, magnesium carbonate, magnesium hydrogen carbonate, hydroxyapatite, boron nitride, hollow silica microspheres (Silica Beads from Maprecos), glass or ceramic microcapsules; Sunsphere L-31 and Sunsphere H-31 sold by Asahi Glass; Chemicelen sold by Asahi Chemical; composites of silica and of titanium dioxide, for instance the TSG series sold by Nippon Sheet Glass, and mixtures thereof.

Among the organic fillers that may be used in the compositions according to the invention, mention may be made of polyamide powders (Nylon® Orgasol from Atochem), poly-β-alanine powder and polyethylene powder, polytetrafluoroethylene powders (Teflon®), N-lauroyl-L-lysine, starch, tetrafluoroethylene polymer powders, hollow polymer microspheres such as Expancel (Nobel Industrie), precipitated calcium carbonate, magnesium carbonate, magnesium hydrogen carbonate, metal soaps derived from organic carboxylic acids containing from 8 to 22 carbon atoms, and for example from 12 to 18 carbon atoms, for example zinc stearate, magnesium stearate or lithium stearate, zinc laurate or magnesium myristate, Polypore® L 200 (Chemdal Corporation), silicone resin microbeads (for example Tospearl® from Toshiba), polyurethane powders, for instance powders of crosslinked polyurethane comprising a copolymer, the said copolymer comprising trimethylol hexyllactone. Inone variant, it may be a polymer of hexamethylene diisocyanate/trimethylol hexyllactone. Such particles are for instance commercially available, for example, under the name Plastic Powder D-400® or Plastic Powder D-800® from the company Toshiki, and mixtures thereof.

A filler may be present in a cosmetic composition in accordance with the invention in a proportion of from about 0.1% to about 30% by weight and for example from about 0.5% to about 15% by weight of filler relative to the total weight of the composition.

A filler that is suitable for the invention may be, for example, a filler whose mean particle size is less than 100 μm or for example is between 1 and 50 μm, for example between 4 and 20 μm.

Additives

A cosmetic composition according to the invention may, furthermore, also comprise any additive usually used in the field under consideration, selected from the group consisting of film-forming agents and, where appropriate, auxiliary film-forming agents, gums, semi-crystalline polymers, gelling agents, emulsifiers and, where appropriate, co-emulsifiers, antioxidants, essential oils, preserving agents, fragrances, cosmetic active agents, neutralizers, moisturizers, antiseptics, vitamins such as vitamin B3 or E and derivatives thereof, and anti-UV agents, and mixtures thereof.

As cosmetic active agents that may be used in the compositions of the invention, mention may be made of moisturizers (polyols, for instance glycerol), vitamins (C, A, E, F, B or PP), essential fatty acids, essential oils, ceramides, sphingolipids, sunscreens that are liposoluble or in the form of nanoparticles, and specific skin-treating active agents (protective agents, antibacterial agents, anti-wrinkle agents, etc.). These active agents may be used, for example, in concentrations of from 0 to 20% and for example from 0.001% to 15% relative to the total weight of the composition.

A person skilled in the art can adjust the nature and amount of the additives present in the compositions in accordance with the invention by means of routine operations, such that the cosmetic properties and the viscosity properties desired for these compositions are not thereby affected.

According to one embodiment, a composition in accordance with the invention may comprise, as ester of polyol(s) and of fatty diacid dimer, a dilinoleyl diol dimer/dilinoleic dimer copolymer (or INCI name: dilinoleyl dimerdilinoleate dimer) and, as polyamide mixture, a stabilized hydrogenated C₃₆ diacid/ethylenediamine condensate, esterified with stearyl alcohol (molecular weight: about 4000) (Uniclear 100 VG®).

According to one embodiment variant, a composition according to the invention may also comprise hydrophobic fumed silica particles, surface-treated with dimethylsilane (Aerosil R972®).

A subject of the present invention is also the use of at least one polyamide with an average molecular mass of less than 100 000, comprising a) a polymer backbone with hydrocarbon-based repeating units containing at least one amide function, b) optionally a pendent fatty chain and/or at least one terminal fatty chain, which is optionally functionalized, containing from 6 to 120 carbon atoms, and being linked to these hydrocarbon-based units, as an agent for structuring at least one fatty phase comprising at least one ester of polyol(s) and of fatty diacid dimer, or an ester thereof.

A subject of the present invention is also a method for the preparation of a cosmetic composition comprising at least one step of structuring at least one fatty phase comprising at least one ester of polyol(s) and of fatty diacid dimer, or an ester thereof, with at least one polyamide with an average molecular mass of less than 100 000, comprising a) a polymer backbone with hydrocarbon-based repeating units containing at least one amide function, b) optionally a pendent fatty chain and/or at least one terminal fatty chain, which is optionally functionalized, containing from 6 to 120 carbon atoms, and being linked to these hydrocarbon-based units as a structuring agent.

In one variant, such a composition may have a hardness of less than or equal to 30 g, measured at about 20° C.

According to one embodiment, the ester of polyol(s) and of fatty diacid dimer used in the use in accordance with the invention may be as defined previously.

According to one embodiment, the polyamide used in the use in accordance with the invention may be as defined previously.

According to one embodiment, a cosmetic composition according to the invention may be prepared according to a process comprising at least the steps consisting in:

a) heating a mixture of at least one ester of polyol(s) and of fatty diacid dimer, or an ester thereof, and of at least one polyamide to the melting point of the said polyamide, for instance in the presence of other compounds of the type such as oil and/or wax, and

b) adding, where appropriate, the other ingredients of the type such as pulverulent materials and excipients, for instance the fumed silica particles, while keeping the mixture obtained in step a) stirred to obtain a homogeneous composition.

The composition may be in a cast form, for example in the form of a stick or wand, in the form of a soft paste in a heating bag, or in the form of a dish.

For example, it may constitute a cast foundation, a cast makeup rouge or eyeshadow, which is for instance coloured, a lipstick, a lip gloss or a concealer product.

A composition according to the invention may especially be in the form of a makeup and/or care composition for the lips, for example a lipstick, a lip balm or a lip gloss.

As indicated previously, a cosmetic composition in accordance with the invention may have transparency and translucency properties.

For the purposes of the invention, this transparency and translucency property means that a coat of the composition of a given thickness allows a portion of visible light to pass through.

If this portion of visible light is scattered, the composition will be defined as being a translucent composition, and if, on the other hand, it is not scattered, then the composition will be defined as being a transparent composition.

The present invention is illustrated with the aid of the examples below. These examples do not in any way limit the present invention.

### EXAMPLE 1

A makeup product of “gloss” type for the lips is prepared, the composition of which is as follows:

| Ingredient | Amount |
|---|---|
| Tridecyl trimellitate | 34.07 |
| Dilinoleyl diol dimer/dilinoleic dimer copolymer | 20 |
| (Lusplan DD-DA7 from Nippon Fine Chemical) | |
| Hydrogenated isoparaffin (6-8 mol of isobutylene) | 34.07 |
| Stabilized hydrogenated C₃₆ diacid/ethylenediamine | 0.4 |
| condensate, esterified with stearyl alcohol (molecular | |
| weight of about 4000) (Uniclear 100 VG ® from | |
| Arizona Chemical) | |
| Hydrophobic fumed silica surface-treated with | 10.5 |
| dimethylsilane (Aerosil R 972 from Degussa) | |
| Fragrance | 0.5 |
| Di-tert-butyl-4-hydroxytoluene | 0.06 |
| Mixture of isopropyl, isobutyl and n-butyl p- | 0.4 |
| hydroxybenzoates (40/30/30) | |
| Total | 100 |

The procedure used is as follows.

The ester of polyol and of fatty diacid dimer (dilinoleyl diol dimer/dilinoleic dimer copolymer), the polyamide (Uniclear 100), the oils, the fatty substances and the preserving agents are mixed together at the melting point of the polyamide, i.e. about 105° C.

Once the mixture is homogeneous and the polyamide has fully melted, the fragrance and the fumed silica particles are gradually added while maintaining the temperature at 100-105° C. with a Rayneri blender.

The mixture is stirred until homogenization is complete and a translucent mixture is obtained.

For greater transparency, this mixture may be subjected to Rayneri blending under vacuum to remove the air bubbles that may be present in the finished product.

The composition is then poured into a mould.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A cosmetic composition for making up and/or caring for keratin materials, combining
at least one ester of polyol(s) and of fatty diacid dimer, or an ester thereof, with
at least one polyamide with an average molecular mass of less than 100,000, comprising a) a polymer backbone with hydrocarbon-based repeating units containing at least one amide function, b) optionally a pendent fatty chain and/or at least one terminal fatty chain, which is optionally functionalized, containing from 6 to 120 carbon atoms, and being linked to these hydrocarbon-based units.

2. The composition according to claim 1, said composition also comprising fumed silica particles.

3. The composition according to claim 1, said composition having a hardness of less than or equal to about 30 g measured at about 20° C.

4. The composition according to claim 3, in which the hardness is about 15 g, measured at about 20° C.

5. The composition according to claim 1, in which said ester has a molecular weight ranging from about 2000 to about 25,000 g/mol.

6. The composition according to claim 1, in which the fatty acid is a C₈ to C₃₄ unsaturated fatty acid.

7. The composition according to claim 6, in which the unsaturated fatty acid is selected from the group consisting of undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid and arachidonic acid, and mixtures thereof.

8. The composition according to claim 1, in which the diacid dimer is derived from the dimerization of linoleic acid.

9. The composition according to claim 1, in which the polyol is a diol.

10. The composition according to claim 9, in which the diol is selected from the group consisting of a fatty alcohol dimer, a monoglycerol or polyglycerol, a C₂-C₄ monoalkylene or polyalkylene glycol, 1,4-butanediol and pentaerythritol.

11. The composition according to claim 9, in which the diol is derived from the hydrogenation of a diacid dimer of at least one unsaturated fatty acid.

12. The composition according to claim 1, in which said ester is chosen from the esters of formula (I), (II) or (IV) below:
R₃—OCO—R₁(—COO—R₂—OCO—R₁)ₙ—COO—R₃ (I)
in which:
COR₁CO represents a fatty diacid dimer residue,
OR₂O represents a fatty alcohol dimer residue,
OR₃ represents a hydrocarbon-based monoalcohol residue, and
n is an integer ranging from 1 to 15, or 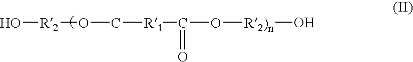
in which:
n is an integer ranging from 1 to 15,
COR′₁CO represents a fatty diacid dimer residue,
OR′₂O represents a diglyceryl residue of general formula (III) below: 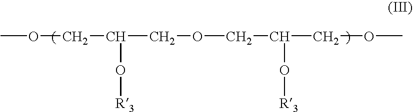
in which:
R′₃ represents H or OR′₃ represents a fatty acid residue, or a mixture thereof, or
HO—R₁″—(—OCO—R₂″—COO—R₁″—)ₕ—OH (IV)
in which:
OR₁″O represents a diol dimer residue obtained by hydrogenation of a dimerdilinoleic acid,
COR₂″CO represents a fatty diacid dimer residue, and
h represents an integer ranging from 1 to 9.

13. The composition according to claim 12, in which:
COR₁CO represents a dimerdilinoleate residue,
OR₂O represents a dimerdilinoleyl residue, and
OR₃ represents a hydrocarbon-based monoalcohol residue selected from the group consisting of behenyl, isostearyl and phytosteryl residues, and mixtures thereof.

14. The composition according to claim 12, in which:
COR₁CO represents a dimerdilinoleate residue, and
the fatty acid residue featured by OR′₃ is an isostearyl residue.

15. The composition according to claim 12, in which COR₂″CO represents a dilinoleate dimer residue.

16. The composition according to claim 1, in which said ester is selected from the group consisting of the esters having the following INCI nomenclature: polyglyceryl-2 isostearate/dimerdilinoleate copolymer, bis-behenyl/isostearyl/phytosteryl dimerdilinoleyl dimerdilinoleate, dimerdilinoleyl dimerdilinoleate, and mixtures thereof.

17. The composition according to claim 1, in which the ester of polyol(s) and of fatty diacid dimer, or an ester thereof, is present in a content ranging from about 1% to about 95% by weight relative to the total weight of the composition.

18. The composition according to claim 1, in which the fatty chains of said polyamide represent from 40% to 98% of the total number of amide units and of fatty chains.

19. The composition according to claim 1, in which the average molecular mass of the polyamide ranges from 1000 to 100,000 g/mol.

20. The composition according to claim 1, in which the terminal fatty chain(s) is (are) bonded to the backbone via ester groups.

21. The composition according to claim 1, in which the fatty chain(s) contain(s) from 12 to 68 carbon atoms.

22. The composition according to claim 1, in which the polyamide is of formula (V) below: 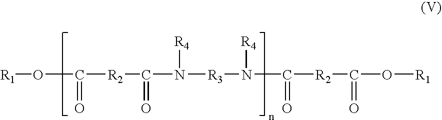
in which:
n denotes a whole number of amide units such that the number of ester groups represents from 10% to 50% of the total number of ester and amide groups;
each of the symbols R₁ independently denotes an alkyl or alkenyl group containing at least 4 carbon atoms;
each of the symbols R₂ independently represents a C₄ to C₄₂ hydrocarbon-based group, on condition that 50% of the groups R₂ represent a C₃₀ to C₄₂ hydrocarbon-based group;
each of the symbols R₃ independently represents an organic group containing at least two carbon atoms, hydrogen atoms and optionally one or more oxygen or nitrogen atoms;
and each of the symbols R4 independently represents a hydrogen atom, a C₁-C₁₀ alkyl group or a direct bond to R₃ or to another R₄ such that the nitrogen atom to which R₃ and R₄ are both bonded forms part of a heterocyclic structure defined by R₄-N-R₃, with at least 50% of the R₄ representing a hydrogen atom.

23. The composition according to claim 22, in which R₁ is a C₁₂-C₂₂ alkyl group.

24. The composition according to claim 22, in which 50% of the R₂ are hydrocarbon-based groups containing from 30 to 42 carbon atoms.

25. The composition according to claim 1, in which the polyamide is present in a content ranging from about 0.01% to about 20% by weight relative to the total weight of the composition.

26. The composition according to claim 1, in which the polyamide is selected from the group consisting of copolymers of C₃₆ diacids condensed onto an ethylenediamine with a weight-average molecular mass of about 6000, and mixtures thereof.

27. The composition according to claim 2, in which the fumed silica particles are selected from the group consisting of hydrophilic fumed silica particles and hydrophobic fumed silica particles.

28. The composition according to claim 2, in which the fumed silica particles are present in a content ranging from about 1% to about 30% by weight relative to the total weight of the composition.

29. The composition according to claim 1, also comprising at least one additional fatty phase selected from the group consisting of a liquid fatty phase and a solid fatty phase, and mixtures thereof

30. The composition according to claim 1, said composition comprising a wax content of less than or equal to 5% by weight relative to the total weight of the composition.

31. The composition according to claim 1, also comprising at least one dyestuff.

32. The composition according to claim 1, said composition being anhydrous.

33. The composition according to claim 1, said composition being a makeup and/or care composition for the lips.

34. The composition according to claim 1, said composition being in the form of a gloss.

35. The composition according to claim 1, said composition being in a compact form.

36. A method for the preparation of a cosmetic composition comprising at least one step of structuring at least one ester of polyol(s) and of fatty diacid dimer, or an ester thereof, with at least one polyamide with an average molecular mass of less than 100,000, comprising a) a polymer backbone with hydrocarbon-based repeating units containing at least one amide function, b) optionally a pendent fatty chain and/or at least one terminal fatty chain, which is optionally functionalized, containing from 6 to 120 carbon atoms, and being linked to these hydrocarbon-based units, as a structuring agent.

37. The method according to claim 36, in which said ester has a molecular weight ranging from about 2000 to about 25,000 g/mol.

38. The method according to claim 36, in which the fatty chains of said polyamide represent from 40% to 98% of the total number of amide units and of fatty chains.

39. A process for making up and/or caring for keratin materials, comprising at least one step that consists in applying to at least part of said keratin materials a composition as defined according to claim 1.
